(19) United States
(12) Patent Application Publication
(10) Pub. No.: US 2009/0130742 A1
(43) Pub. Date: May 21, 2009

(54) VESSEL AND SYSTEM FOR BIOLOGICAL REGENERATION OF ION EXCHANGE AND ABSORPTIVE MEDIA

(75) Inventors: Samuel Frisch, Manalapan, NJ (US); Michael A. Del Vecchio, Flemington, NJ (US); Robert Loudon, Howell, NJ (US)

Correspondence Address: RATNERPRESTIA, P.O. BOX 980, VALLEY FORGE, PA, 19482, US

(73) Assignee: Shaw Environmental & Infrastructure, Inc., Lawrenceville, NJ (US)

(21) Appl. No.: 11/941,630

(22) Filed: Nov. 16, 2007

(51) Int. Cl. C12M 1/18; C12M 1/16; C12S 9/00; C12M 1/08

(52) U.S. Cl. 435/264; 435/299.1; 435/295.1

(57) ABSTRACT

A system and method for biological regeneration of ion exchange and absorptive media including a vessel configured to contain a bed of contaminated media particles. The vessel includes a first region configured to receive biological regenerating fluid for contacting media particles in the first region at a first volumetric flowrate sufficient to produce a shear force high enough to reduce bio-film thickness on the media particles; a second region configured to receive a portion of biological regenerating fluid from the first region, wherein the portion of biological regenerating fluid in said second region has a second volumetric flowrate lower than the first volumetric flowrate; and a third region configured to receive another portion of biological regenerating fluid from the first region, wherein the another portion of biological regenerating fluid in the third region has a third volumetric flowrate lower than the second volumetric flowrate.

### FIELD OF THE INVENTION

The invention relates to a vessel and system for the biological regeneration of ion exchange and absorptive media. More specifically, the invention relates to a contacting system for the regeneration of ion exchange and/or absorptive media utilizing biological degradation as the method of regeneration.

### BACKGROUND OF THE INVENTION

In, for example, treatment of drinking water to render it potable, ion exchange and absorptive media are used to remove harmful contaminants. For example, perchlorate found in drinking water is a contaminant know to pose serious health risks. One method of removing contaminants, such as perchlorates, from drinking water is by treating the contaminated water with ion exchange and/or absorptive media. Eventually, in any treatment using ion exchange and absorptive media system, the media becomes exhausted and is no longer effective in removing the harmful contaminants. Thus, there is a need for a system and method for treating contaminated wastewaters to address the regeneration of media.

### SUMMARY OF THE INVENTION

In one aspect, the invention provides a system for biological regeneration of ion exchange and absorptive media. The system comprises a vessel configured to contain a bed of contaminated media particles and having a first region, a second region and a third region. The first region is configured to receive a biological regenerating fluid for contacting media particles at a first volumetric flowrate. This first volumetric flowrate is sufficient to produce a shear force high enough to reduce bio-film thickness on the media particles, while not so high as to cause significant attrition of the media. The second region is configured to receive a portion of biological regenerating fluid from the first region, wherein the portion of biological regenerating fluid in the second region has a second volumetric flowrate lower than the first volumetric flowrate. The third region configured to receive another portion of biological regenerating fluid from the first region, wherein the another portion of biological regenerating fluid in the third region has a third volumetric flowrate lower than the second volumetric flowrate.

In another aspect, the invention provides a method of biologically regenerating ion exchange and/or absorptive media. In one step, the method comprises feeding a biological regenerating fluid into a first region of a vessel containing contaminated media particles at a first volumetric flowrate sufficient to produce a shear force high enough to reduce bio-film thickness on the media particles. In another step, the method comprises dividing the biological regenerating fluid from the first region into portions. In yet another step, the invention comprises directing one of the portions of biological regenerating fluid into a second region of the vessel at a second volumetric flowrate lower than the first volumetric flowrate. In further step, the invention comprises directing another one of the portions of biological regenerating fluid into a third region of the vessel at a third volumetric flowrate lower than the second volumetric flowrate.

In an embodiment according to aspects of the invention, the invention provides a system for biological regeneration of ion exchange and absorptive media. According to the embodiment, the invention includes a vessel configured to contain a bed of contaminated media particles. The system further includes a draft tube disposed within the vessel and having an inlet spaced above a bottom of the vessel and an outlet disposed proximate to a top of the bed of media particles, the draft tube configured to receive a biological regenerating fluid for contacting the contaminated media particles. The embodiment also includes a substantially annular region longitudinally disposed at an elevation above the draft tube outlet and below a liquid deflector being disposed above the draft tube outlet, and radially disposed between an outside wall of the draft tube and an inside wall of the vessel, the substantially annular region configured to receive a portion of biological regenerating fluid from the draft tube. Further, the system includes an upper region of the vessel disposed above the deflector and comprising an area defined by a substantially full inside diameter of the vessel, the upper region configured to receive another portion of biological regenerating fluid from the draft tube.

In another embodiment according to aspects of the invention, the invention provides a system for biological regeneration of ion exchange and absorptive media. The system includes a vessel configured to contain a bed of contaminated media particles. The system further includes a central passage disposed within the vessel and having an inlet disposed at a top of the vessel and an outlet disposed at an elevation above and spaced apart from a bottom of the vessel, the central passage configured to receive biological regenerating fluid for contacting the contaminated media particles. The system according to this embodiment also includes a first substantially annular region configured to receive biological regenerating fluid from the central passage, the first substantially annular region longitudinally disposed from a bottom of the vessel to a top of the media bed, and radially disposed between an outside wall of the central passage and an inside wall of the vessel. Further, the system includes a second substantially annular region longitudinally disposed (1) from a top of the media bed to an elevation below first gas deflector disposed in an upper region of the vessel, (2) between an outside wall of the central passage and an inside wall of the vessel, the second substantially annular region configured to receive a portion of biological regenerating fluid from the first substantially annular region. The system according to this embodiment also includes an upper region of the vessel longitudinally disposed above the gas deflector and between the outside wall of the central passage and the inside wall of the vessel. The upper region is configured to receive another portion of biological regenerating fluid from the first substantially annular region.

In yet another embodiment according to aspects of the invention, the invention provides a system for biological regeneration of ion exchange and absorptive media. The system includes a vessel configured to contain a bed of contaminated media particles. The system further includes an outer central passage positioned within the vessel and has an inlet disposed at a top of the vessel and an outlet disposed in an upper region of the vessel. The outer central passage is configured to receive a biological regenerating fluid for contacting the contaminated media particles. The system according to this embodiment also includes a first substantially annular region longitudinally disposed between a bottom region of the vessel and a top of the media bed, and radially disposed between an outer wall of the inner central passage and an inner wall of the vessel. The first substantially annular region is configured to receive a portion of biological regenerating fluid from the outer central passage. Further, the system includes an inner central passage having an inlet at a bottom region of the vessel and an outlet disposed at the top of the vessel, the inner central passage being configured to receive the portion of biological regenerating fluid from the first substantially annular region. The system according to this embodiment also includes a second substantially annular region longitudinally disposed between a top of the media bed and a top of the vessel and radially disposed between the outer wall of the outer central passage, a portion of the inner central passage and the inner wall of the vessel. The second substantially annular region is configured to receive another portion of biological regenerating fluid from the outer central passage.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is a block diagram of an exemplary embodiment of a system for biological regeneration according to the present invention.

FIG. 2 is a flow diagram of an exemplary embodiment of a system for biological regeneration according to the present invention.

FIG. 3 is a cross-sectional view of an exemplary embodiment of a system of the present invention wherein a vessel includes a draft tube.

FIG. 3A is an exploded partial cross-section view of the system of FIG. 3.

FIG. 3B is a cross-sectional view of the system shown in FIG. 3, illustrating Zones A, B and C in the vessel.

FIG. 4 is a cross-sectional view of another exemplary embodiment of a system of the present invention wherein a vessel includes an draft tube having a skirt.

FIG. 4A is an exploded partial cross-section view of the system of FIG. 4.

FIG. 5 is cross-sectional view of an yet another exemplary embodiment of a system of the present invention wherein a vessel includes a well-screen in a dished-shaped bottom head.

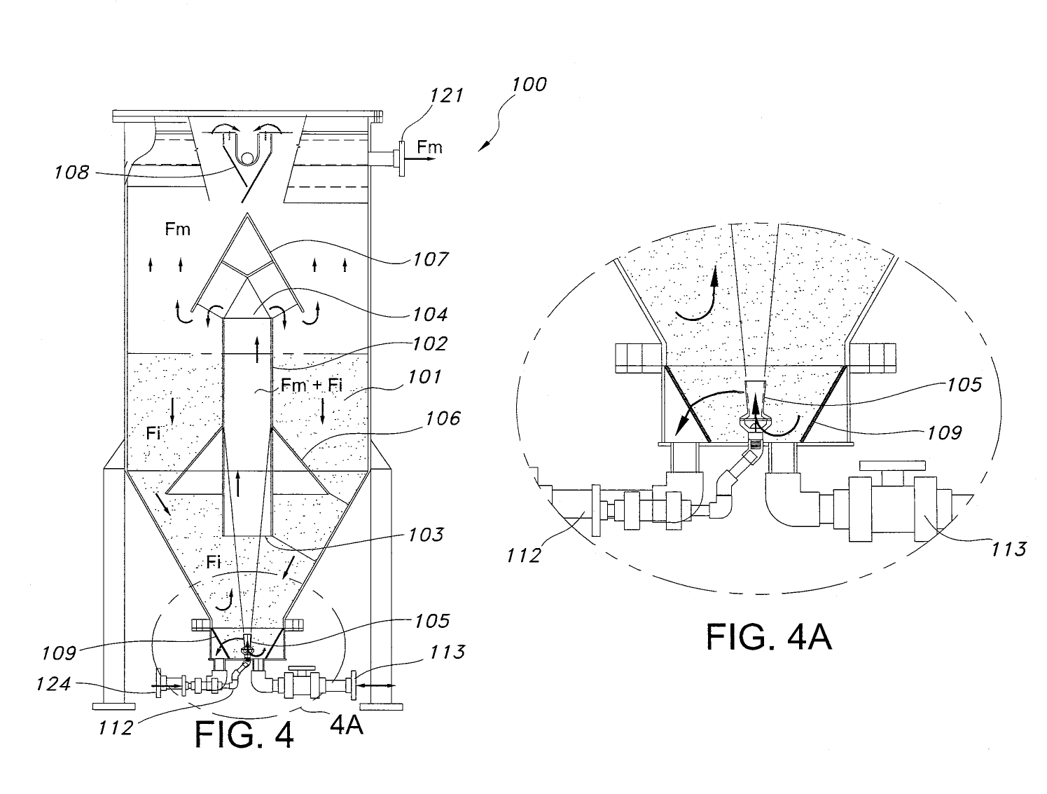

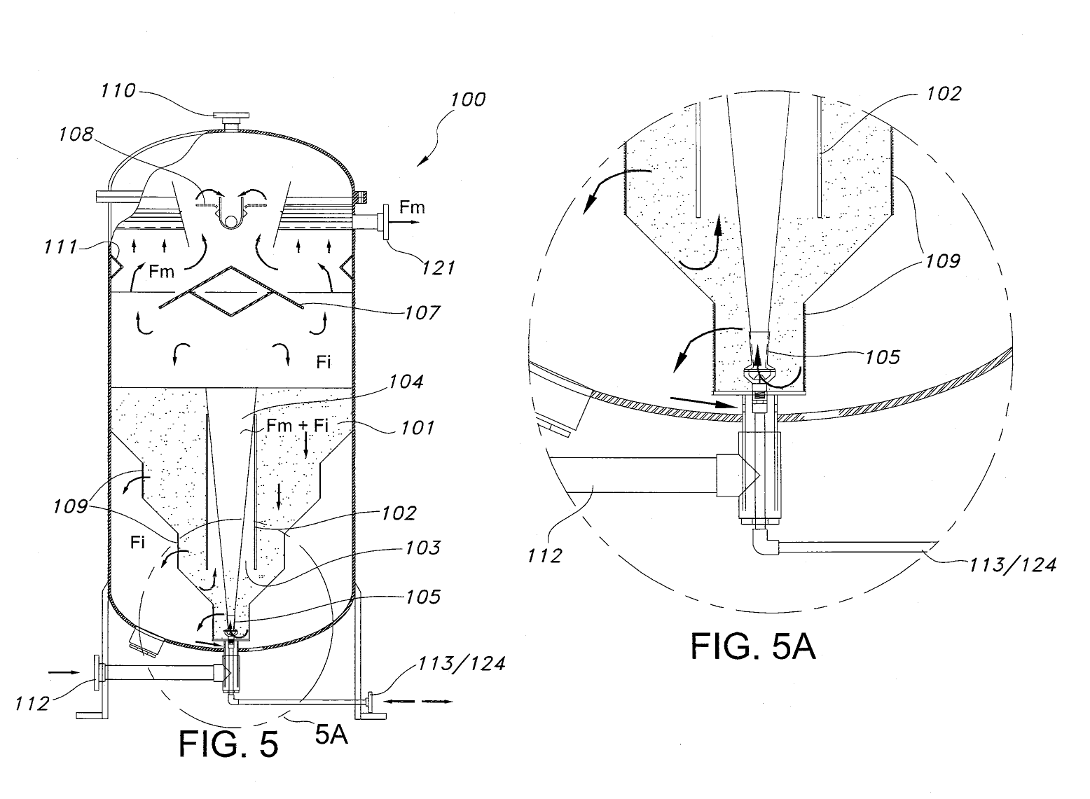

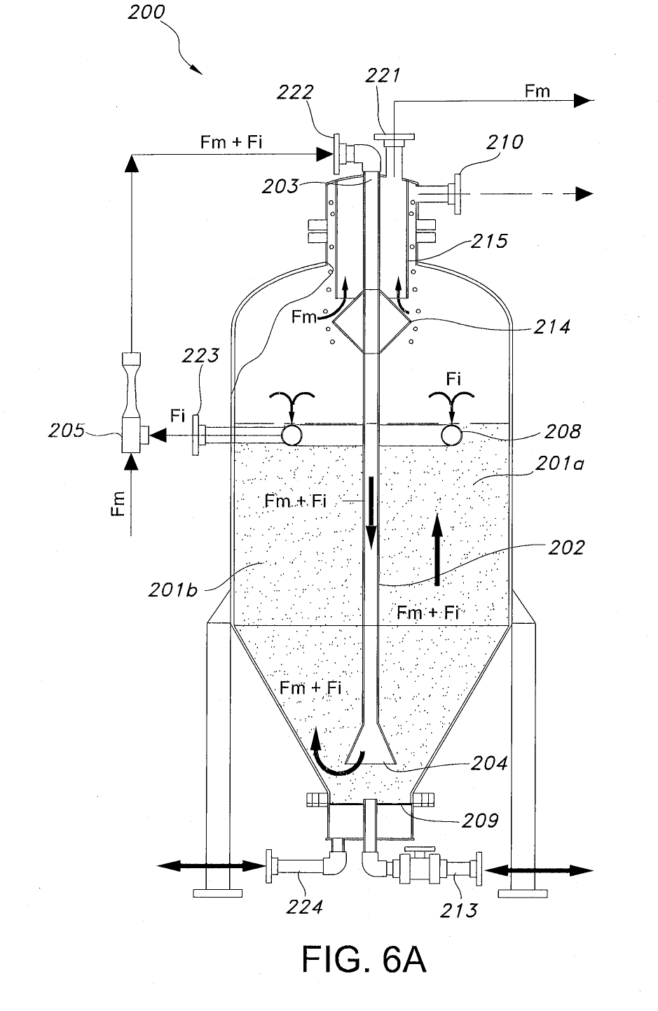

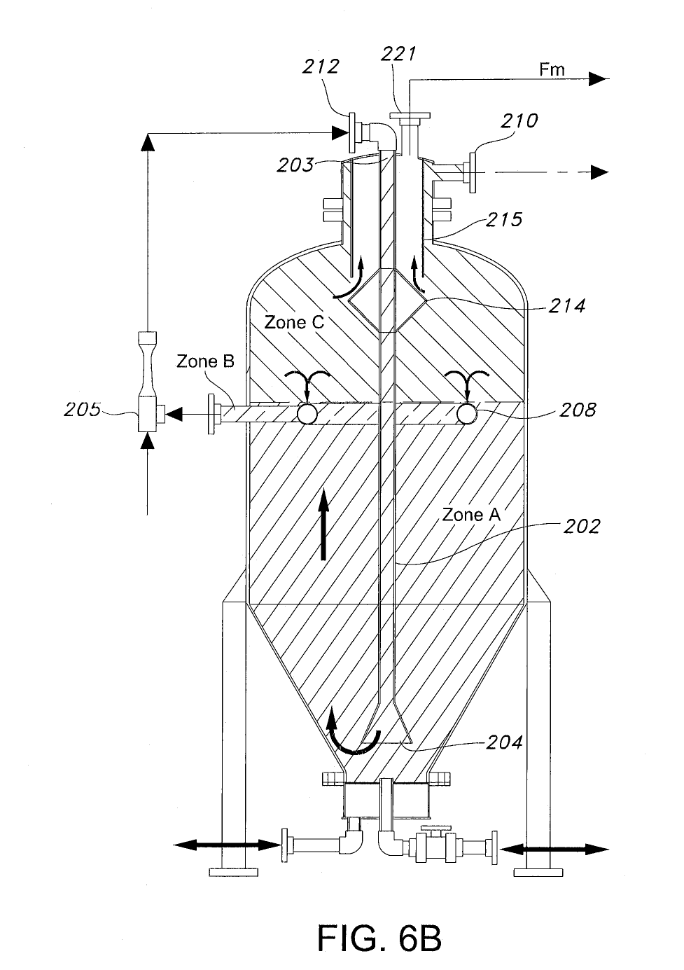

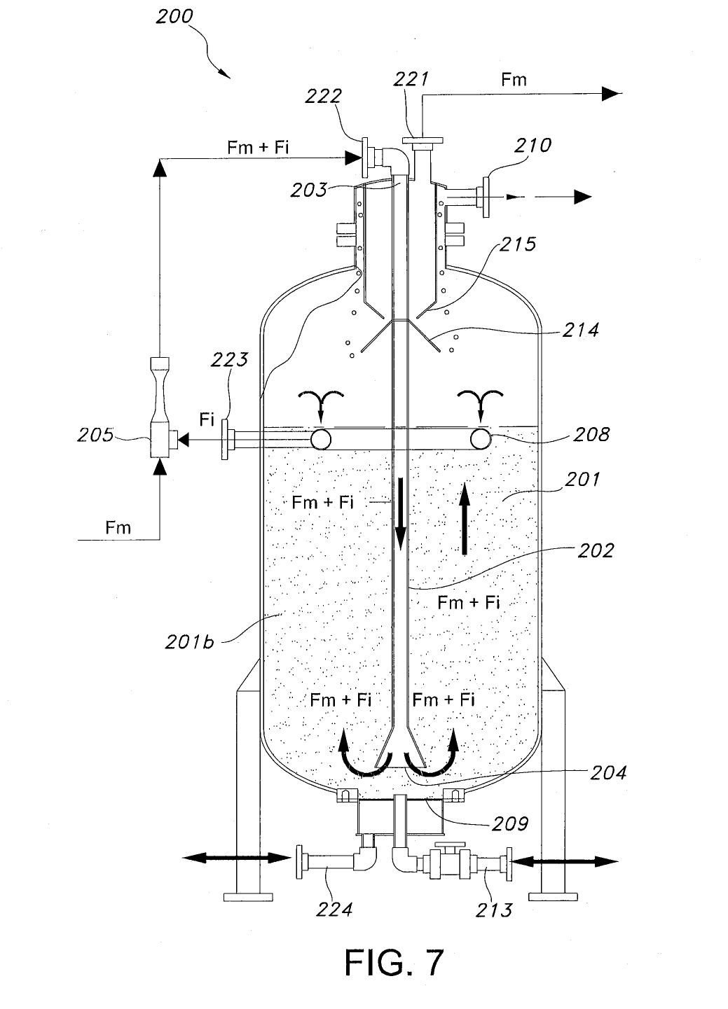

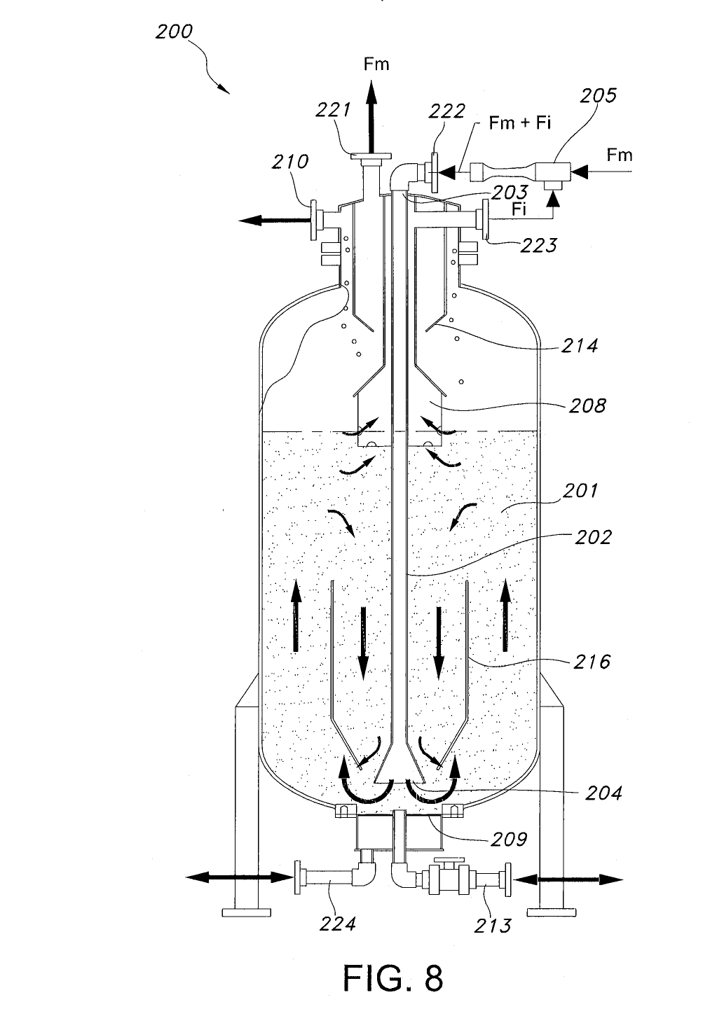

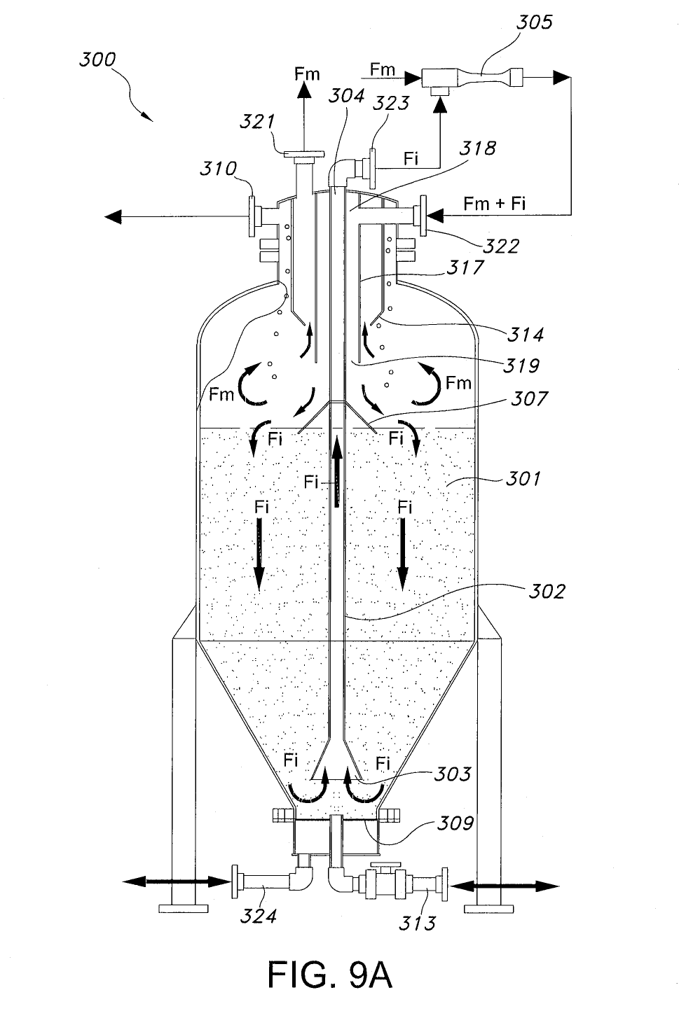

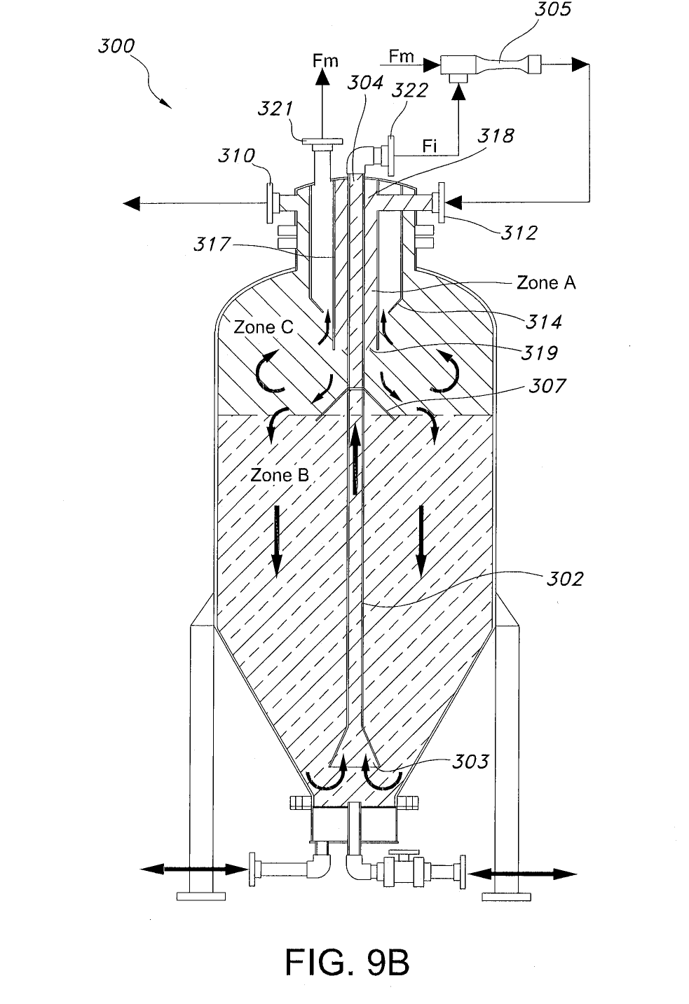

FIG. 4A is an exploded partial cross-section view of the system of FIG. 5.

FIG. 6A is cross-sectional view of a still yet another exemplary embodiment of a system of the present invention wherein a vessel includes an end-to-end closed cone-shaped gas deflector.

FIG. 6B is a cross-sectional view of the system of FIG. 6A, illustrating Zones A, B and C in the vessel.

FIG. 7 is a cross-sectional view of a another exemplary embodiment of a system of the present invention wherein a vessel includes an inverted cone-shaped gas deflector.

FIG. 8 is a cross-sectional view of another exemplary embodiment of a system of the present invention wherein a vessel includes a swiss-cheese shaped liquid collector.

FIG. 9A is a cross-sectional view of yet another exemplary embodiment of a system of the present invention wherein a vessel includes an outer and an inner central pipe.

FIG. 9B is a cross-sectional view of the system shown in FIG. 9A, illustrating Zones A, B and C in the vessel.

### DETAILED DESCRIPTION OF THE INVENTION

This invention generally relates to a method for regenerating exhausted media in which one of the steps includes a biological regeneration step. In such a biological regeneration step of the regeneration process it is preferred for the contaminant that is contained within the ion exchange or adsorptive media to diffuse into the bulk liquid phase on the outside of the particle in order for the biological degradation to occur. The bioactivity in the bulk liquid phase continually destroys the contaminant and therefore the concentration of the contaminant in the liquid phase remains very low. Diffusion of the contaminant from the interior of the media particle to its surface and finally into the bulk liquid phase is motivated by a concentration gradient. As contaminants are removed from the media a counter flow of water and dissolved minerals and ions diffuses into the media particles in order to replace the void space left by the contaminants and, in the case of ionic constituents, to maintain electrical neutrality. It is preferred in this process to improve the rate of diffusion and to promote complete regeneration of all the media particles.

The regeneration process typically causes gases to evolve. For example, carbon dioxide and nitrogen, among other gases, may be generated due to the bio-conversion from dissolved organic constituents and/or nitrates. The gas will often evolve as a fine bubble on the surface of the media particle.

Further, biomass will tend to grow within the contacting system as a film on the surface of the media particle. This is because of the tendency of biomass to stick to surfaces and because constituents diffusing out of the media particle are a nutrient source for the microorganisms. The combination of gas bubbles and biomass growth on the surface of the media particles increases the particle buoyancy. In a contacting system that is flowing and relies on gravity as the means of separation of the media particles from the bulk fluid (such as in a biological fluidized bed reactor) the increased buoyancy of the particle often reduces the efficiency of the gravity separation and causes loss of media from the vessel. Use of a positive means of retaining the media, such as a screen or filter, though an optional alternative, can result in pluggage with biomass or significant maintenance.

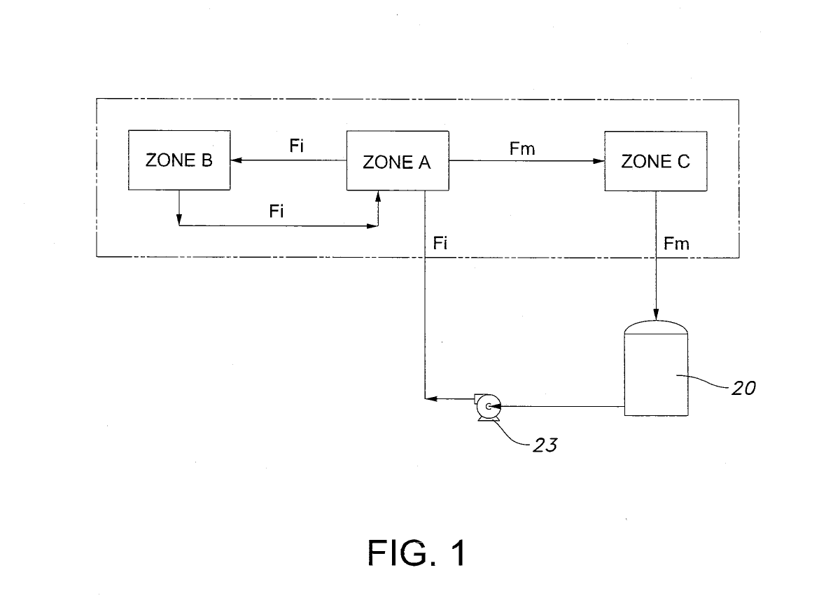

Generally, the present invention provides a system and process for the biological regeneration of ion exchange and/or absorptive media, such as ion exchange resin, granular activated carbon, activated alumina, synthetic adsorbents and zeolites. In one aspect, the invention provides a system for biological regeneration of ion exchange and absorptive media comprising a vessel configured to contain a bed of contaminated media particles. As represented by the block diagram in FIG. 1, which illustrates an exemplary embodiment of the invention, the apparatus comprises a vessel 1 having three regions, the first region designated as Zone A, a second region designated as Zone B and a third region designated as Zone C. The first region, Zone A, is configured to receive a biological regenerating fluid, such as a bio-suspension, for contacting media particles in the first region. The bio-suspension comprises a aqueous suspension of microorganisms or biomass. The fluid contacting the media particles in the first region has a first volumetric flowrate sufficient to produce a shear force high enough to reduce bio-film thickness on the media particles. As shown in the block diagram of FIG. 1, the flow into Zone A is equal to the total flow of the system, which is equal to the motive flow, Fₘ, plus the induced flow, Fᵢ. As illustrated in FIG. 1, the flow from Zone A is divided into two portions, the portions being fed to Zones B and Zone C, respectively.

One of the portions of the flow is defined as being equal to the induced flow, Fᵢ, and enters the second region, Zone B, as illustrated in FIG. 1. This second region, Zone B, is configured to receive a portion of biological regenerating fluid from the first region, wherein the portion of biological regenerating fluid in the second region has a second volumetric flowrate lower than the first volumetric flowrate. As shown in FIG. 1, the flow exiting from Zone B is equal to the induced flow, Fᵢ. This flow is recirculated back to Zone A.

The third region of the vessel, Zone C, is configured to receive another portion of biological regenerating fluid from the first region. This portion of biological regenerating fluid introduced into the third region has a third volumetric flowrate which is lower than the second volumetric flowrate. This flow into Zone C is equal to the motive flow, Fₘ, as shown in FIG. 1. The flow, Fₘ, from Zone C is recirculated back to the first zone via pumping device 23, such as a recirculation pump, and combined with the induced flow, Fᵢ, from Zone B.

In order to achieve a shear force high enough to reduce bio-film thickness on the media, but not enough shear to cause attrition of the media, it is expected that the induced flow would be equal to the motive flow or as high as four times the motive flow. The shear force may be adjusted, for example, by varying the pressure of the motive flow, throttling the discharge from pumping device 23 and/or varying the speed of the pumping device 23.

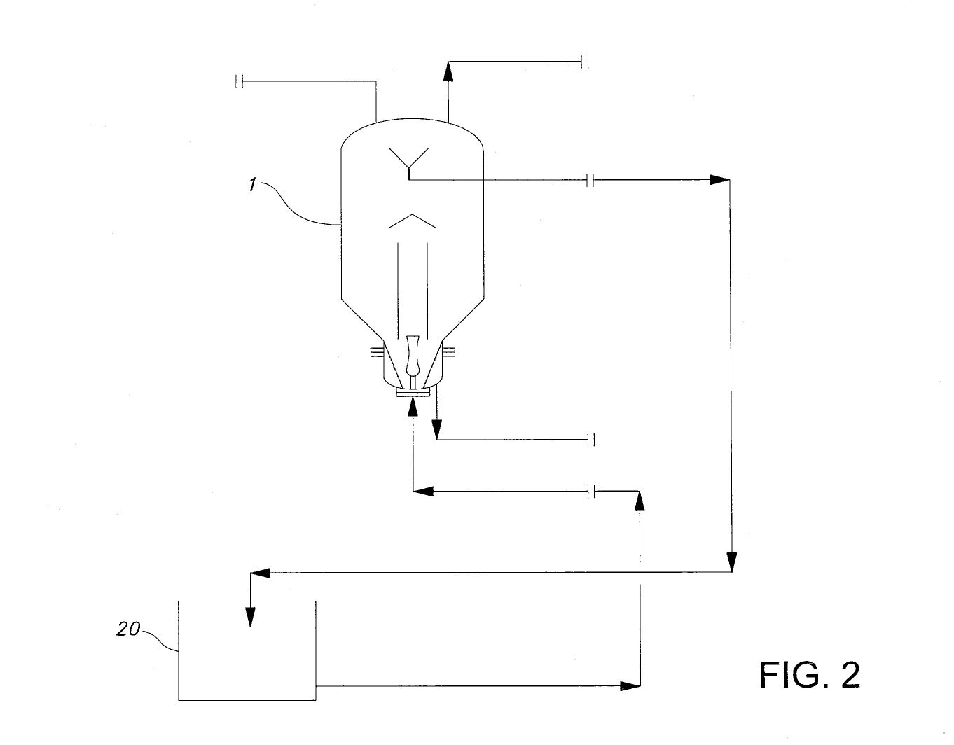

As shown schematically in the flow diagram of FIG. 2, the system according to an exemplary embodiment generally includes vessel 1 containing ion exchange and/or absorptive media into which is fed a biological regenerating fluid from a tank 20, such as a fermentor tank or a bioactivity support vessel. The fluid is injected into vessel 1, shown in FIG. 2 as the flow entering vessel 1 at the bottom of the vessel 1. The vessel 1 internals, discussed in detail below, are configured to provide improved ion exchange resin and/or absorptive media regeneration. The biological regenerating fluid is recovered and recirculated back to tank 20. Gases evolved as a product of the reaction of the biological regenerating fluid with the contaminated ion exchange and/or absorptive media are released from vessel 1 via a vent disposed at the top of tank 1. Once the regeneration process is complete, the tank 1 is drained of biological regenerating fluid out the bottom of the tank 1 and further steps as required for regeneration of the media, as are known to those of ordinary skill in the art, can be performed.

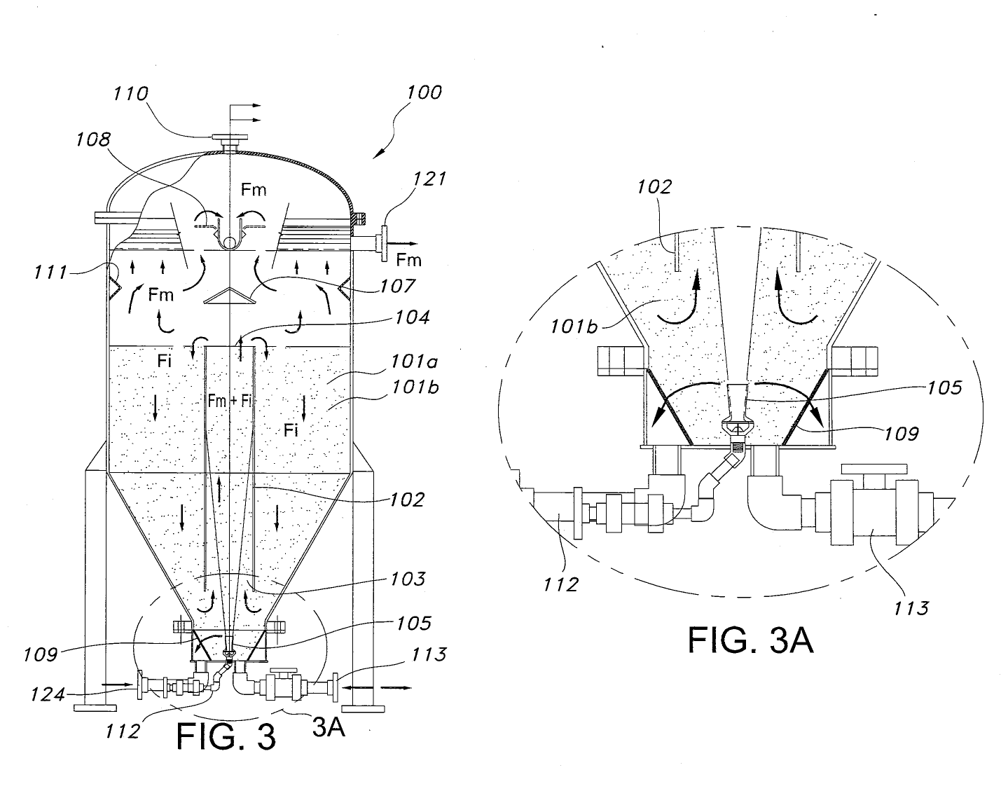

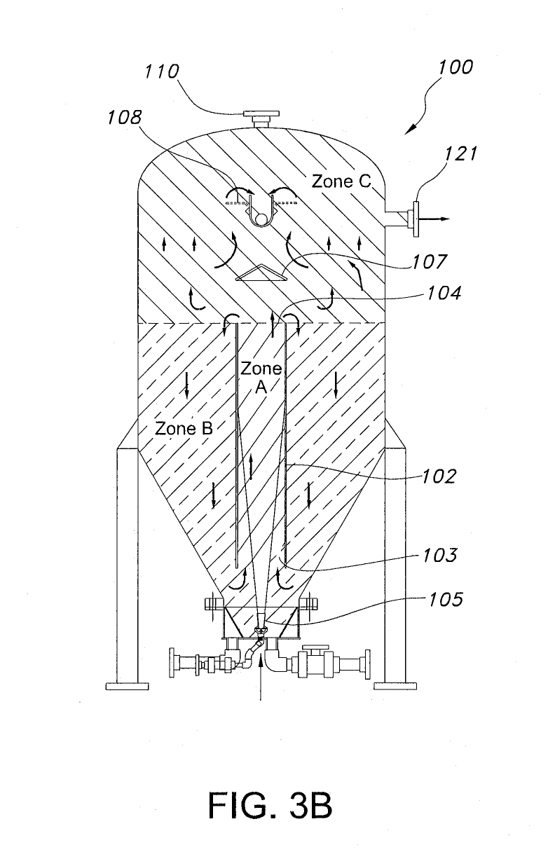

Further details of exemplary embodiments of the invention are now provided, with reference to FIGS. 3-9. In an embodiment according to the first aspect of the invention, as shown in FIG. 3, the invention provides an apparatus for biological regeneration of ion exchange and absorptive media comprising a vessel 100 configured to contain a bed 101a of contaminated media particles 101b. FIG. 3B illustrates the location of each of Zones A, B and C within the vessel. The first region, according to this embodiment, comprises a central passage, such as a draft tube, 102 disposed within the vessel 100. The draft tube 102 has an inlet 103 spaced above a bottom region of the vessel 100 and an outlet 104 disposed proximate to a top of the bed 101a of media particles 101b. For example, the draft tube outlet 104 may be disposed at, above or below the bed 101a of media particles 101b, as shown in FIGS. 3, 4 and 5, respectively. The draft tube 102 is configured to receive a biological regenerating fluid for contacting the media particles 101b in the draft tube 102 at a first volumetric flowrate sufficient to produce a shear force high enough to reduce bio-film thickness on the media particles. In this embodiment, this first volumetric flowrate is equal to the total of the motive flow, Fₘ, of the fluid plus the induced flow of the fluid, Fᵢ. Thus, the total flow is equal to Fₘ plus Fᵢ.

The draft tube 102 optionally may be configured to receive the portion of the biological regenerating fluid of the second region (described below) at the inlet 103 for recirculation back into the draft tube 102. Further, the draft tube 102 receives the biological regenerating fluid from a feed device 105, such as a tank mixing eductor (TME), an impeller arrangement or a gas airlift device. Preferably, the feed device 105 is a TME, for example, of the type manufactured by Penberthy, Inc. of Prophetstown, Ill. The feed device 105, as shown in FIG. 3, is disposed at the bottom of the vessel 100 and is spaced below the draft tube 102.

As shown in FIG. 3, vessel 100 includes liquid deflector 107 disposed at an elevation above and spaced apart from the draft tube outlet 104. The fluid deflector 107 can be, for example, an angled liquid deflector positioned to divide the biological regenerating fluid into two portions. The first portion of the stream is directed downwardly toward a bottom of the vessel 100. The second portion of the biological regenerating fluid is directed upwardly toward a top the vessel 100. The deflector 107 may optionally include a hole positioned to prevent the buildup of gases evolved from the media after contact with the biological regenerating fluid. The first portion of the stream from draft tube 102 transitions into the second region of the vessel which includes the substantially annular region longitudinally disposed at an elevation above the draft tube outlet 104 and below liquid deflector 107. The substantially annular region is radially disposed between an outside wall of the draft tube 102 and an inside wall of the vessel 100. The substantially annular region is configured to receive a portion of biological regenerating fluid from the draft tube 102. The portion of biological regenerating fluid in the substantially annular region has a second volumetric flowrate lower than the first volumetric flowrate. The flow in the second region includes a volumetric flowrate equal to the induced flow, Fᵢ, of the fluid.

Vessel 100 further includes a third region disposed above the liquid deflector 107. This third region includes an upper region of the vessel 100 which comprises an area defined by a full inside diameter of vessel 100. The third region is configured to receive the second portion of biological regenerating fluid from the draft tube 102. This second portion has a third volumetric flowrate lower than the second volumetric flowrate. The flow in the third region may include a volumetric flowrate equal to the motive flow, Fₘ, of the fluid.

Optionally, the draft tube 102 is provided with an external baffle arrangement, or skirt, 106, such as that shown in FIG. 4. Skirt 106 aids in distributing the media particles 101b as they fall from liquid deflector 107 to the bottom of the tank. As shown in FIG. 4, the skirt 106 forces the media 101b away from the center of the vessel 100 to prevent the media particles 101b from failing directly down to the bottom center of vessel 100 and to help improve flow distribution of the media 101 with the biological regenerating fluid. A further optional feature of vessel 100 includes wall baffles 111, as shown in FIG. 5, disposed along the inside wall of vessel 100. These wall baffles 111, although shown disposed at substantially the same elevation as liquid deflector 107, may be positioned above or below the liquid deflector 107. Preferably, the wall baffles are positioned above the liquid deflector. Wall baffles 111 help promote uniform flow distribution of the biological regenerating fluid so as to optimize the disengagement of entrained media in any upward flowing biological regenerating fluid to prevent the media 101b from being carried upward, and potentially out of the vessel 100.

As shown in the embodiments of FIGS. 3, 4 and 5, vessel 100 further includes a collection system, or collector, 108 configured to disengage media particles and gas bubbles from the portion of biological regenerating fluid in the upper portion of the vessel 100. The biological regenerating fluid is recovered and returned, for example, to a fermentor tank, such as that illustrated in the flow diagram of FIG. 2, where it can be recharged and recirculated back to the vessel 100 for further regeneration of the media particles 101b. Further, the vessel 100 includes a resin retaining screen 109 located at a bottom of the vessel 100, as shown in FIGS. 3A, 4A and 5A. A vent 110 for releasing gases evolved as a bio-conversion product between the contaminated media and the biological regenerating fluid in the upper region of vessel 100.

According to the embodiments shown in FIGS. 3, 4 and 5, in the first region the horizontal cross-sectional area is based on the diameter of the draft tube 102. The bio-suspension total fluid flowrate in the first region, which includes the total of the motive flow, Fₘ, plus the induced flow, Fᵢ, is the highest flow within the vessel 100. In this first region, the high velocity causes shear to be applied to the media particles 101b, causing bio-films on the surface of the media 101b to be controlled and maintained in a very thin state. This minimizes the resistance to diffusion that would otherwise be caused by the bio-film. Additionally, the overall particle density is not adversely affected and its buoyancy is not increased. In other words, the volumetric flowrate in this region is intended to be within a range that is high enough to minimize bio-film thickness on the media 101b, but not so high as to cause undue attrition or breakage of the media particles 101b. The flowrate is adjustable by adjustment of the motive flow, Fₘ, through the feed device 105, exemplified in FIGS. 3, 4 and 5 as an eductor.

In this embodiment of the invention, the second region, Zone B, comprises the substantially annular region between the draft tube 102 and the inner wall of the vessel 100, as shown in FIGS. 3, 4 and 5. A portion of bio-suspension and substantially all of the media particles 101b enter the second region. The biological regenerating fluid flow in this second region is downward and equal to the induced flowrate, Fᵢ, of the fluid. Substantially all the media 101b in this region flows toward the bottom of the vessel 100 where it is again lifted, with the flow entering the draft tube 102 from feed device 105 into the draft tube 102. In this second region, it is believed that the induced flow, Fᵢ, is approximately equal to two times the motive flow, Fₘ. However, this ratio can vary with the motive flowrate, Fₘ, the design of the feed device 105, the characteristics of the media 101b, and the design and size of the draft tube 102. For example, it is estimated that for a 42 inch diameter vessel operating at 10 psi and having an 8″ diameter draft tube, for a total flow of 21.9 gpm, the induced flow would be 14.6 gpm and the motive flow would be 7.3 gpm. In other embodiments, the induced flow may be equal to the motive flow or as high as four times the motive flow.

The third region in this embodiment is located above the draft tube 102. The flow in this region is upward and equal to the motive flow, Fₘ, that is fed by feed device 105. It is believed that the flow rate is approximately one-third of the total flow through the draft tube 102, which is equal to the motive flow, Fₘ, and the induced flow, Fᵢ. The area of vessel 100 in this third region is based on substantially the full vessel diameter and is the largest cross-sectional passage area that the fluid flows through inside the vessel 100. Thus, the combination of low flow and high area result in a very low velocity in the third region. Additionally, the buoyancy of the media particles is minimized as a result of the very thin biofilm layer or layers resulting from the shear applied in the draft tube 102. Accordingly, the separation of the media 101b from the biological regenerating fluid bio-suspension occurs at a very high efficiency. The bio-suspension exits the vessel 100 via a collection system, such as a collector, 108 that is designed to disengage any remaining media particles 101b and gas bubbles from the stream.

As shown in FIGS. 3 and 4, the bottom of the vessel 100 is optionally frusto-conical in shape and includes a screened drain 112 and an unscreened drain 113 through which water may be removed or added to the vessel 100. The screened drain 109 allows fluid to be drained from the vessel 100 while preventing the media particles 101b from being carried out of the vessel 100 with the fluid being drained. By virtue of the screened and unscreened connections at the bottom of the vessel 100 optimal flow patters can be established for both regeneration and cleaning modes of operation. These flow patterns result in a uniform expansion of the bed 101a of media particles 101b. Further, this feature further enables the vessel 100 to function efficiently in washing and rinsing the media particles 101b before and after the bio-regeneration functions. Flow may be introduced into the screened drained in order to uniformly expand the bed without creating the circular motion resulting from the eductor. Among the operations that utilize this pattern of flow is a backwash operation that efficiently washes suspended solids from the media. The screen also allows for uniform packed bed down flow operation, such as a rinsing or a draining operation.

As noted above, vessel 100, as shown in FIGS. 3 and 4, includes a frusto-conical shaped bottom. The purpose of this shape is to funnel the media 101b toward the bottom center of the vessel 100 below the draft tube inlet 103 so that the media 101b is recirculated back up through the draft tube 102. This configuration helps to prevent the formation of “dead areas,” i.e. areas of low flow where the contaminated media 101b can be trapped and not adequately exposed to the biological regenerating fluid. In a variation of this embodiment, as shown in FIG. 5, the vessel 100 can optionally include a dished or rounded bottom. However, to prevent “dead areas,” the vessel 100 includes a resin retaining screen 109 configured to funnel the media 101b toward the draft tube inlet 103, as exemplified by the resin retaining well screen 109 in the figure.

The vessel 100 may be made from corrosion resistant material, such as fiberglass, or the vessel may be made from steel that has been coated with a corrosion resistant material. In certain embodiments, the resin retaining screen 109 may be a metallic screen made from exotic metals, such as MONEL® or duplex alloys.

The vessel 100 can optionally be operated at atmospheric pressure. Additionally, the operating pressure within the vessel 100 may be maintained at a level that prevents, inhibits or reduces gas that is converted from the dissolved liquid phase into a free gas state. As liquid exits the vessel 100, via outlet 121, the pressure may be reduced to atmospheric conditions to liberate the gases that have formed in the contacting (regeneration) vessel 100. Agitation may optionally be used outside of the contacting vessel 100 to further enhance liberation of gases. After the excess gases are removed from the biological regenerating fluid, the fluid may be re-pressurized inside the contacting vessel 100. As it enters the contacting vessel 100, the bio-suspension may thus be maintained in a substantially sub-saturated state, thus allowing the bio-suspension to absorb and accumulate more gas in a dissolved liquid state without evolution of free gas.

In further embodiments according to the first aspect, as shown in FIGS. 6, 7 and 8, a system is provided for biological regeneration of ion exchange and/or absorptive media comprising a vessel 200 configured to contain a bed 201a of contaminated media particles 201b. In a first region, Zone A, the vessel 200 includes a central passage 202 having an inlet 203 disposed at a top of the vessel 200 and an outlet 204 disposed above and spaced apart from the bottom of the vessel 200. Zone A, as well as Zones B and C, are illustrated in FIG. 6B. The central passage 202 is configured to receive biological regenerating fluid for contacting the contaminated media particles 201b. The biological regenerating fluid is fed to the vessel 200 via inlet 212 via eductor 205. The central passage, such as a central pipe, 202 can optionally include a trumpet-shaped outlet 204, which allows the vessel 200 to be designed with a larger annulus at the bottom of the vessel 200 in order to avoid “dead areas” and ensure proper movement of all media within the vessel and establish more uniform upflow within the annular space 201. By combining the trumpet-shaped outlet 204 of central passage 202 with a dished-headed bottom, the vessel height may also be reduced.

Further, the first region also includes a substantially annular region outside the central passage 202. More specifically, this substantially annular region is longitudinally disposed from a bottom of the vessel 200 to a top of the bed 201a of media particles 201b, and radially disposed between an outside wall of the central passage 202 and an inside wall of the vessel 200. The substantially annular region is configured to receive a portion of biological regenerating fluid from the central passage 202. The flow in this first region includes the total flow of the system, i.e. the motive flow, Fm, plus the induced flow, Fi.

In the second region, the vessel 200 is provided with a second substantially annular region longitudinally disposed from a top of the bed 201a of media particles 201b to an elevation below a first gas deflector 214 being disposed at an elevation in an upper region of the vessel 200, and radially disposed at an elevation between an outside wall of central passage 202 and an inside wall of the vessel 200. The second substantially annular region is configured to receive a first portion of biological regenerating fluid from the first substantially annular space.

As shown in FIGS. 6, 7 and 8, the second region of the system further comprises a liquid collection system, such as a collector, 208 configured to collect a first portion of the biological regenerating fluid flowing from the first substantially annular region for recirculation, via outlet 223 to the feed device 205. This provides the advantage of having the media 201b subjected to additional shear as the biological regenerating fluid is recirculated through the feed device 205. This is in contrast to embodiments in which the fluid is recirculated via a draft tube. The collection system 208 can be, for example, a drilled pipe-ring collector, as shown in FIGS. 6 and 7, or a “swiss-cheese” can type collector, as shown in FIG. 8. The first portion of biological regenerating fluid in the second substantially annular region has a second volumetric flowrate lower than the first volumetric flowrate. The flowrate in the second region is equal to the induced flow, Fᵢ.

In these embodiments, the third region of vessel 200 further includes an upper region of the vessel 200, longitudinally disposed above the gas deflector 212 and between an outer wall of the central passage 202 and an inner wall of the vessel 200. The upper region is configured to receive a second portion of biological regenerating fluid from the first substantially annular region. The second portion of the biological regenerating fluid has a third volumetric flowrate lower than the second volumetric flowrate. The flow in this third region is equal to the motive flow, Fₘ, of the fluid, which is less than the induced flow, Fᵢ. The biological regenerating fluid exits the vessel 200 via outlet 221 for recirculation back to the vessel 200.

In these embodiments, the bed of media particles 201b is fluidized as the biological regenerating exiting from the outlet 204 of central passage 202 flows upward through the bed of media particles 201b, as shown in FIGS. 6, 7 and 8.

The vessel 200 according to these embodiments optionally comprise a frusto-conical shaped bottom, as shown in FIG. 6, or, alternatively, a dish-shaped bottom head, as shown in FIGS. 7 and 8. The vessel 200 further comprises a feed device 205, such as an eductor, configured to feed the biological regenerating fluid into the vessel 200 containing contaminated media particles 201b at the first volumetric flowrate. The feed device 205 may optionally be disposed externally of the vessel 205.

The system according to these embodiments as shown, for example, in FIGS. 6, 7 and 8, further provides a first gas deflector 214 positioned to disengage gas bubbles and media 201 from the second portion of the biological regenerating fluid. The first gas deflector 214 may be, for example, an end-to-end closed cone-shaped baffle, as shown in FIG. 6. Alternatively, the first gas deflector 214 may be a cone-shaped baffle, as shown in FIG. 7, in which the gas deflector optionally includes a hole to prevent buildup of gases. The first gas deflector 214 is optionally disposed on the central passage 202 in the upper region of the vessel 200.

In an embodiment of the invention, such as shown in FIGS. 6 and 7, the vessel may also be provided with a second gas deflector 215 disposed at an elevation above the first gas deflector 214. The second gas deflector 215 can include a baffle having a frusto-conical shape with the lower portion directed toward a central passage 202 of the vessel 200. The baffle optionally is not frusto-conical shape and can instead optionally be shaped as a cylinder. The first and second gas deflectors 214 and 215, respectively, each help to prevent media 201b from being vented with the gases.

Optionally, the vessel 200 may further include a lower baffle arrangement 216 in a bottom region of the vessel 200, as shown in FIG. 8, to help prevent the biological regenerating fluid from short-circuiting the media 201b along the walls of vessel 200. As shown, lower baffle arrangement 216 has a substantially frusto-conical shape with a large opening at the top for receiving biological regenerating fluid and media 201b and a narrower opening at the bottom. The bottom of the lower baffle arrangement 216 is disposed at an elevation at or just above the central passage outlet 204 and is centered about a lower section of the central passage 202, as shown in FIG. 8. The baffle arrangement 216 improves mixing by creating a venturi effect proximate to the central passage outlet 204 and the bottom of vessel 200. As shown in FIG. 8, the lower baffle arrangement 216 creates loop of flow to increase contact time and mixing of the resin with the biological regenerating fluid.

Further, the vessel 200 includes, as shown in FIGS. 6, 7 and 8, a resin retaining screen 209 disposed at a bottom of the vessel 200. In addition, the vessel 200 further includes a vent 211 positioned to release gas evolved as a bio-conversion product from the contaminated media 201b and the biological regenerating fluid.

In yet another embodiment, exemplified by FIG. 9, the invention provides a system for biological regeneration of ion exchange and/or absorptive media. The system according to such an embodiment provides a vessel 300 configured to contain a bed 301a of contaminated media particles 301b. As shown in FIG. 9, the system includes a first zone, Zone A, including an outer central passage 317 within the vessel 300, having an inlet 318 disposed at a top of the vessel 300 and an outlet 319 disposed in an upper region of the vessel 300. The outer central passage 317 is configured to receive a biological regenerating fluid, for example from feed device 305, such as an eductor, for contacting contaminated media particles 301b at a first volumetric flowrate sufficient to produce a shear force high enough to reduce bio-film thickness on the media particles 301b. The feed device may optionally be disposed externally of the vessel 300, and provides the biological regenerating fluid to the vessel 300 via inlet 322. This flow is equal to the total flow of the system, which includes the motive flow, Fₘ, and the induced flow, Fᵢ.

The system as shown in FIG. 9 includes an angled liquid deflector 307 configured to receive biological regenerating fluid from the outer central passage 317 and to divide the biological regenerating fluid into a first portion and a second portion. The deflector 307 is oriented to direct the first portion of the biological regenerating fluid downwardly toward a bottom of the vessel 300 and into a second region, Zone B, of the vessel 300. The deflector 307 directs a second portion the biological regenerating fluid upwardly toward a top the vessel 300 and into a third region of the vessel 300.

As shown in FIG. 9, the second region includes a first substantially annular region disposed longitudinally between a bottom region of the vessel 300 and a top of the bed 301a of media particles 301b. The first substantially annular region is radially disposed between an outer wall of inner central passage 302 and an inner wall of the vessel 300. The first substantially annular region is configured to receive a first portion of biological regenerating fluid from the outer central passage 317. Inner central passage 302 has an inlet 303 at a bottom region of the vessel 300 and an outlet 304 disposed at the top of the vessel 300. The inner central passage 302 is configured to receive the first portion of biological regenerating fluid from the first substantially annular region, and may optionally be included with a trumpet-shaped inlet 303. The flow in the first substantially annular region and the central passage 302 is equal to the induced volumetric flowrate, Fᵢ. The flow from inner central passage 302 exits the vessel 300 via outlet 323 for recirculation to feed device 305.

The vessel 300 further includes in the third region, Zone C, a second substantially annular region disposed longitudinally between a top of the bed 301a of media particles 301b and the top of the vessel 300. Each of Zones A, B and C, as they are substantially located in vessel 300, are illustrated in FIG. 9B as represented by hashed lines for each zone. This second substantially annular region is radially disposed between an outer wall of the outer central passage 317, a portion of the inner central passage 302 and the inner wall of the vessel 300 outer wall, as illustrated in FIG. 9. The second substantially annular region is configured to receive a second portion of biological regenerating fluid from the outer central passage 317. The flowrate in the second substantially annular region is equal to the motive flow, Fₘ, which is less than the induced flow, Fᵢ. The flow from the second substantially annular region exits vessel 300 via outlet 321 and is recirculated back to the vessel 300 via feed device 305.

Further, as shown in FIG. 9, the vessel 300 may comprise a frusto-conical shaped bottom. Alternatively, the vessel 300 may comprise a dished-shaped bottom. As shown in FIG. 9, the vessel 300 includes a resin retaining screen 309 at a bottom of the vessel 300. Although the resin retaining screen 309 is shown as a flat screen, it is contemplated that the screen 309 may have other configurations.

The system according to this embodiment provides a vent 310 configured to release gas evolved as a bio-conversion product between the contaminated media 301b and the biological regenerating fluid. To ensure that gas bubbles and media particles 301b are disengaged from the biological regenerating fluid, the vessel 300 may also be provided with a gas deflector 315 disposed in an upper region of the vessel 300. The gas deflector 315 can include a baffle having a frusto-conical shape with the lower portion directed toward the outer central passage 317 of the vessel 300. Alternatively, the gas deflector may also have a cylinder shaped configuration rather than a substantially frusto-conical shape.

In this embodiment, the vessel configuration allows the bed to be packed and the vessel to operate with a downward flow. The overall vessel volume may be reduced because the media in this embodiment is not expanded.

In another aspect, the invention provides a method of biologically regenerating ion exchange and/or absorptive media. The method includes feeding a biological regenerating fluid into a first region of a vessel containing contaminated media particles. The biological regenerating fluid is fed at a first volumetric flowrate sufficient to produce a shear force high enough to reduce bio-film thickness on the media particles. In a next step, the method includes dividing the biological regenerating fluid from the first region into portions and directing one of the portions of biological regenerating fluid into a second region of the vessel at a second volumetric flowrate lower than the first volumetric flowrate. The method further includes directing another one of the portions of biological regenerating fluid into a third region of the vessel at a third volumetric flowrate lower than the second volumetric flowrate. In embodiments according to this aspect, gases are caused to evolve from the contaminated media that are produced from bio-conversion of the biological regenerating fluid and the contaminated media.

In an embodiment according to this aspect, the method includes directing the portion of the biological regenerating fluid having the second volumetric flowrate downward toward a bottom of the vessel. A further step of this embodiment may comprise directing the second portion of the biological regenerating fluid upward toward a top of the vessel.

In another embodiment according to this aspect, the method optionally comprises the step of recirculating the biological regenerating fluid of the second portion to the first region.

The system and method for regenerating ion exchange and absorptive media according to embodiments of the present invention generally provides the advantage that bio-films on the surface of the media particles are controlled and are maintained in a very thin state due to an optimum application of sheer force on the media particles. This minimizes the resistance to diffusion that would otherwise be caused by the bio-film. Additionally, the overall particle density is not adversely affected and its buoyancy is not increased.

Further, according to exemplary embodiments of this invention, gases are inhibited from forming and are separated from the particles and removed from the bulk fluid flow before they can adversely affect the ability of the particle to be separated from the bulk liquid bio-suspension and reduce regeneration time. This is accomplished by application of an optimum amount of sheer to the particles and by changes in direction of flow within the vessel as the liquid bio-suspension and fluidized media flow through the vessel.

Another advantage of exemplary embodiments of the present invention is that the embodied configurations provide rapid mix zones, with the media placed into circulation causing substantially all particles to be equally exposed to the flowing bio-suspension. Thus, the system according to exemplary embodiments of this invention, has no “dead areas” or areas where media may be trapped and not adequately exposed to the biological regenerating fluid, or bio-suspension. This is preferred because any media that would otherwise be trapped in “dead” or low flow areas would not be fully regenerated and would cause premature leakage in the adsorptive or ion exchange systems into which the regenerated media would subsequently be placed. Thus, it is believed that near complete regeneration of substantially all media with no “dead” or low activity zones is attained.

A still further advantage of exemplary embodiments of the present invention is that its configuration provides very low up-flow velocity of the bio-suspension in the upper regions of the vessel to separate out the media particles. This greatly reduces the chance for media carryover due to free gas bubbles on the media, thus preventing, or at least reducing, media loss.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A system for biological regeneration of ion exchange and absorptive media comprising:
a vessel configured to contain a bed of contaminated media particles and having a first region, a second region and a third region;
said first region being configured to receive a biological regenerating fluid for contacting media particles in said first region at a first volumetric flowrate sufficient to produce a shear force high enough to reduce bio-film thickness on said media particles;
said second region being configured to receive a portion of biological regenerating fluid from said first region, wherein the portion of biological regenerating fluid in said second region has a second volumetric flowrate lower than said first volumetric flowrate; and
said third region being configured to receive another portion of biological regenerating fluid from said first region, wherein the another portion of biological regenerating fluid in said third region has a third volumetric flowrate lower than said second volumetric flowrate.

2. The system of claim 1 wherein said vessel further comprises a feed device configured to feed said biological regenerating fluid into said vessel containing said contaminated media at said first velocity.

3. The system of claim 2 wherein said feed device is an eductor.

4. The system of claim 3 wherein said eductor is located at a bottom of said vessel.

5. The system of claim 3 wherein said eductor is located externally of said vessel.

6. The system of claim 1 wherein said vessel further comprises a frustoconical-shaped bottom.

7. The system of claim 1 wherein said vessel further comprises a dished-shaped bottom.

8. The system of claim 1 wherein said first region comprises a central passage positioned to receive said biological regenerating fluid from a feed device disposed at a bottom of said vessel.

9. The system of claim 8 wherein said central passage is defined by a draft tube having an inlet spaced above said feed device and an outlet disposed at a top of said media bed.

10. The system of claim 9 wherein said draft tube is positioned to receive said second portion of said biological regenerating fluid from said second region at said inlet and to recirculate said portion of the biological regenerating fluid.

11. The system of claim 8 wherein said central passage includes an inlet disposed at a top of said vessel and an outlet disposed at an elevation above and spaced apart from a bottom of said vessel.

12. The system of claim 11 wherein said central passage comprises a trumpet-shaped outlet.

13. The system of claim 8 wherein said central passage includes an external baffle disposed on an outside thereon.

14. The system of claim 1 wherein said vessel includes an angled liquid deflector positioned to receive biological regenerating fluid from said first region and to divide said biological regenerating fluid into said portions.

15. The system of claim 14 wherein said deflector is oriented to direct said portion of biological regenerating fluid downwardly toward a bottom of said vessel and said another portion of biological regenerating fluid upwardly toward a top said vessel.

16. The system of claim 1 further comprising a collector disposed at an upper region of said vessel and positioned to disengage gas bubbles and media from said another portion of biological regenerating fluid.

17. The system of claim 1 further comprising a first gas deflector positioned to disengage gas bubbles and media from said another portion of said biological regenerating fluid.

18. The system of claim 17 wherein said first gas deflector comprises an end-to-end closed cone shaped baffle.

19. The system of claim 17 wherein said first gas deflector includes a cone-shaped baffle.

20. The system of claim 17 wherein said first gas deflector includes a hole positioned to prevent buildup of gases.

21. The system of claim 17 further comprising a second gas deflector disposed at an elevation above said first gas deflector in a top region of said vessel.

22. The system of claim 21 wherein said second gas deflector comprises a baffle having angles disposed with respect to a center of said vessel.

23. The system of claim 1 further comprising a collector positioned to collect said portion of biological regenerating fluid flowing from said first region for recirculation.

24. The system of claim 23 wherein said collector comprises a drilled pipe-ring collector.

25. The system of claim 23 wherein said collector comprises a swiss-cheese can collector.

26. The system of claim 1 wherein said vessel further comprises a resin retaining screen at a bottom region of said vessel.

27. The system of claim 1 wherein said first volumetric flowrate includes a total volumetric flowrate equal to a volumetric motive flowrate of said fluid and a volumetric induced flow rate of said biological regenerating fluid.

28. The system of claim 1 wherein said second volumetric flowrate is equal to a volumetric induced flowrate of said biological regenerating fluid.

29. The system of claim 1 wherein said third volumetric flowrate is equal to a volumetric motive flowrate of said biological regenerating fluid.

30. The system of claim 1 further comprising a vent disposed at a top of said vessel and positioned to release gases evolved as a bio-conversion product of said contaminated media and said biological regenerating fluid.

31. A method of biologically regenerating ion exchange and absorptive media comprising the steps of:
feeding a biological regenerating fluid into a first region of a vessel containing contaminated media particles at a first volumetric flowrate sufficient to produce a shear force high enough to reduce bio-film thickness on the media particles;
dividing the biological regenerating fluid from the first region into portions,
directing one of the portions of biological regenerating fluid into a second region of the vessel at a second volumetric flowrate lower than said first volumetric flowrate; and
directing another one of the portions of biological regenerating fluid into a third region of the vessel at a third volumetric flowrate lower than said second volumetric flowrate.

32. The method of claim 31 further comprising the step of evolving gases from said contaminated media resulting from bio-conversion of the biological regenerating fluid and the contaminated media.

33. The method of claim 31 further comprising recirculating said biological regenerating fluid of said second portion to said first region.

34. The method of claim 31 further comprising directing the portion of the biological regenerating fluid having the second volumetric flowrate downward toward a bottom of said vessel.

35. The method of claim 34 further comprising directing the another portion of the biological regenerating fluid upward toward a top of said vessel.

36. A system for biological regeneration of ion exchange and absorptive media comprising:
a vessel configured to contain a bed of contaminated media particles;
a draft tube disposed within said vessel and having an inlet spaced above a bottom of said vessel and an outlet disposed proximate to a top of the bed of media particles, said draft tube being configured to receive a biological regenerating fluid for contacting the contaminated media particles;
a substantially annular region longitudinally disposed at an elevation above said draft tube outlet and below a liquid deflector disposed at an elevation above said draft tube outlet, said substantially annular region being radially disposed between an outside wall of said draft tube and an inside wall of said vessel, and said substantially annular region being configured to receive a portion of biological regenerating fluid from said draft tube; and
an upper region of said vessel disposed at an elevation above said deflector and comprising an area defined by a substantially full inside diameter of said vessel, said upper region being configured to receive another portion of biological regenerating fluid from said draft tube.

37. The system of claim 36 wherein said draft tube is positioned to receive said second portion of said biological regenerating fluid from said substantially annular region at said inlet to recirculate said another portion.

38. The system of claim 36 wherein said draft tube receives said biological regenerating fluid from a feed device disposed at a bottom of said vessel.

39. The system of claim 36 wherein said feed device comprises an eductor.

40. The system of claim 36 wherein said draft tube includes a trumpet-shaped inlet.

41. The system of claim 36 wherein said draft tube further comprises an external baffle disposed on an outside thereon.

42. The system of claim 36 wherein said deflector comprises an angled liquid deflector positioned to divide said biological regenerating fluid into said portions.

43. The system of claim 42 wherein said deflector includes a hole configured to prevent buildup of gases.

44. The system of claim 36 wherein said deflector is oriented to direct said portion of said biological regenerating fluid downwardly toward a bottom of said vessel, and said another portion of said biological regenerating fluid upwardly toward an upper region of said vessel.

45. The system of claim 36 further comprising a collector positioned to disengage gas bubbles and media from the another portion of biological regenerating fluid.

46. The system of claim 36 wherein said vessel further comprises a resin retaining screen disposed at a bottom of said vessel.

47. The system of claim 36 further comprising a vent positioned to release gas evolved as a bio-conversion product between said contaminated media and said biological regenerating fluid.

48. A system for biological regeneration of ion exchange and absorptive media comprising:
a vessel configured to contain a bed of contaminated media particles;
a central passage disposed within said vessel and having an inlet disposed at a top of said vessel and an outlet disposed at an elevation above and spaced apart from a bottom of said vessel, said central passage being configured to receive biological regenerating fluid for contacting the contaminated media particles;
a first substantially annular region configured to receive biological regenerating fluid from said central passage, said first substantially annular region longitudinally being disposed from a bottom of said vessel to a top of the media bed and being radially disposed between an outside wall of said central passage and an inside wall of said vessel;
a second substantially annular region longitudinally disposed (1) from a top of the media bed to an elevation below a first gas deflector disposed in an upper region of said vessel, and (2) between an outside wall of central passage and an inside wall of said vessel, said second substantially annular region being configured to receive a portion of biological regenerating fluid from said first substantially annular region;
an upper region of said vessel longitudinally disposed above said gas deflector and between the outside wall of said central passage and the inside wall of said vessel, said upper region being configured to receive another portion of biological regenerating fluid from said first substantially annular region.

49. The system of claim 48 wherein the bed of media particles is fluidized.

50. The system of claim 48 wherein said vessel further comprises a frustoconical-shaped bottom.

51. The system of claim 48 wherein said vessel further comprises a dished-shaped bottom.

52. The system of claim 48 wherein said vessel further comprises a feed device configured to feed said biological regenerating fluid to said central passage.

53. The system of claim 52 wherein said feed device is an eductor.

54. The system of claim 53 wherein said eductor is located externally of said vessel.

55. The system of claim 48 wherein said central passage includes a trumpet-shaped outlet.

56. The system of claim 48 wherein the first gas deflector is positioned to disengage gas bubbles and media from said another portion of said biological regenerating fluid.

57. The system of claim 48 wherein said first gas deflector includes an end-to-end closed cone-shaped baffle disposed on said central passage in said upper region.

58. The system of claim 48 wherein said first gas deflector comprises a cone shaped baffle disposed on said central passage in said upper region.

59. The system of claim 48 further comprising a second gas deflector disposed at an elevation above said first gas deflector in said upper region.

60. The system of claim 59 wherein said second gas deflector comprises a baffle having angles directed downwardly toward said central passage.

61. The system of claim 48 further comprising a liquid collector positioned to collect said portion of the biological regenerating fluid from said first substantially annular region for recirculation.

62. The system of claim 61 wherein said collector comprises a drilled pipe-ring collector.

63. The system of claim 61 wherein said collector comprises a swiss-cheese can collector.

64. The system of claim 48 wherein said vessel further comprises a resin retaining screen disposed at a bottom of said vessel.

65. The system of claim 48 further comprising a vent positioned to release gas evolved as a bio-conversion product from said contaminated media and said biological regenerating fluid.

66. A system for biological regeneration of ion exchange and absorptive media comprising:
a vessel configured to contain a bed of contaminated media particles;
an outer central passage positioned within said vessel and having an inlet disposed at a top of said vessel and an outlet disposed in an upper region of said vessel, said outer central passage configured to receive a biological regenerating fluid for contacting the contaminated media particles;
a first substantially annular region longitudinally disposed between a bottom region of said vessel and a top of said media bed and radially disposed between an outer wall of said inner central passage and an inner wall of said vessel, said first substantially annular region being configured to receive a portion of biological regenerating fluid from said outer central passage;
an inner central passage having an inlet at a bottom region of said vessel and an outlet disposed at the top of said vessel, said inner central passage being configured to receive the portion of biological regenerating fluid from said first substantially annular region; and
a second substantially annular region longitudinally disposed between a top of said media bed and a top of said vessel and radially disposed between the outer wall of said outer central passage, a portion of said inner central passage and the inner wall of said vessel, said second substantially annular region being configured to receive another portion of biological regenerating fluid from said outer central passage.

67. The system of claim 66 wherein said vessel further comprises a feed device configured to feed said biological regenerating fluid into said outer central passage.

68. The system of claim 67 wherein said feed device is an eductor.

69. The system of claim 67 wherein said eductor is disposed externally of said vessel.

70. The system of claim 66 wherein said vessel further comprises a frustoconical-shaped bottom.

71. The system of claim 66 wherein said vessel further comprises a dished-shaped bottom.

72. The system of claim 66 wherein said inner central passage includes a trumpet-shaped inlet.

73. The system of claim 66 wherein the vessel further comprises an angled liquid deflector positioned to receive biological regenerating fluid from said outer central passage and to divide the biological regenerating fluid into said portions.

74. The system of claim 73 wherein said deflector is oriented to direct said portion of biological regenerating fluid downwardly toward a bottom of said vessel and said another portion of said biological regenerating fluid upwardly toward a top said vessel.

75. The system of claim 66 further comprising a gas deflector positioned to disengage gas bubbles and media from another portion of biological regenerating fluid.

76. The system of claim 75 wherein said gas deflector comprises a baffle having angles directed downwardly toward said inner central passage.

77. The system of claim 66 wherein said vessel further comprises a resin retaining screen at a bottom of said vessel.

78. The system of claim 66 further comprising a vent positioned to release gas evolved as a bio-conversion product between said contaminated media and said biological regenerating fluid.
